(19) United States
(12) Patent Application Publication
(10) Pub. No.: US 2006/0069415 A1
(43) Pub. Date: Mar. 30, 2006

(54) Electrical stimulation system, lead, and method providing modified reduced neuroplasticity effect

(75) Inventors: Tracy Cameron, Toronto (CA); Christopher Chavez, Plano, TX (US)

Correspondence Address: FULBRIGHT & JAWORSKI, L.L.P. (ANS), 2200 ROSS AVENUE, SUITE 2800, DALLAS, TX, 75201-2784, US

(73) Assignee: Advanced Neuromodulation Systems, Inc., Plano, TX (US)

(21) Appl. No.: 11/207,086

(22) Filed: Aug. 18, 2005

Related U.S. Application Data
(63) Continuation-in-part of application No. 10/994,008, filed on Nov. 18, 2004.
(60) Provisional application No. 60/523,710, filed on Nov. 20, 2003.
(60) Provisional application No. 60/602,522, filed on Aug. 18, 2004.

(51) Int. Cl. A61N 1/18

(52) U.S. Cl. 607/45

(57) ABSTRACT

According to one aspect, an electrical stimulation system provides modified neuroplasticity effects in a person's nerve tissue. The system includes an electrical stimulation lead adapted for implantation into the person's body for electrical stimulation of target nerve tissue. The stimulation lead includes one or more electrodes adapted to be positioned proximate the target nerve tissue and to deliver electrical stimulation pulses to the target nerve tissue. The system also includes a stimulation source connectable to the stimulation lead and operable to generate the electrical stimulation pulses for transmission to the one or more electrodes of the stimulation lead to cause the one or more electrodes to deliver the electrical stimulation pulses to the target nerve tissue to modify neuroplasticity effects in the target nerve tissue.

### CROSS-REFERENCE TO RELATED APPLICATIONS

This application is a continuation-in-part of U.S. application Ser. No. 10/994,008 filed Nov. 18, 2004, which claims priority to U.S. Application No. 60/523,710 filed Nov. 20, 2003. This application also claims priority to U.S. Provisional Application No. 60/602,522 filed Aug. 18, 2004. All applications are incorporated by reference in their entirety.

### TECHNICAL FIELD

This application relates generally to electrical stimulation of nerve tissue and in particular to an electrical stimulation system, lead, and method providing modified neuroplasticity effects in a person's nerve tissue.

### BACKGROUND

Many people experience adverse conditions associated with functions of the cortex, the thalamus, and other brain structures. Such conditions have been treated effectively by delivering electrical stimulation pulses to one or more target areas of the brain. One method of delivering electrical stimulation pulses to the brain involves inserting an electrical stimulation lead through a burr hole formed in the skull and then positioning electrodes of the stimulation lead in a precise location proximate a target area of the brain to be stimulated such that stimulation of the target area causes a desired clinical effect. For example, one desired clinical effect may be cessation of tremor from a movement disorder such as Parkinson's Disease. A variety of other clinical conditions may also be treated with deep brain stimulation, such as essential tremor, tremor from multiple sclerosis or brain injury, or dystonia or other movement disorders. The electrical stimulation lead implanted in the brain is connected to a stimulation pulse generator implanted at a separate site in the body, such as in the upper chest.

Electrical stimulation may also be applied to nerve tissue in the spinal cord or a peripheral nerve to treat regions of the body affected by chronic pain from a variety of etiologies. According to one technique, a set of efficacious stimulation parameters are determined, the set of parameters is entered into a stimulation system, and the stimulation system is used to electrically stimulate particular nerve tissue in the spinal cord or a peripheral nerve according to the set of parameters. Typically, an implanted stimulation pulse generator transmits electrical stimulation pulses to an implanted electrical stimulation lead according to the set of parameters and, in response, the electrodes of the implanted stimulation lead deliver the electrical stimulation pulses to the particular nerve tissue in the spinal cord or a peripheral nerve. The electrical stimulation pulses stimulate the particular nerve tissue in the spinal cord or a peripheral nerve to cause a subjective sensation of numbness or tingling in the affected region of the body, known as “paresthesia,” which masks or otherwise relieves pain in the affected region. For example, the electrodes may be located external to the dura adjacent particular nerve tissue in the spinal cord that is to be stimulated.

Although electrical simulation of nerve tissue is often an effective treatment, the efficacy of the treatment associated with a particular set of stimulation parameters may decrease in time due to neuroplasticity of the nerve tissue. Neuroplasticity refers to the ability of nerve tissue to dynamically reorganize itself in response to certain stimuli to form new neural connections. This allows the neurons in the nerve tissue to compensate for injury or disease and adjust their activity in response to new situations or changes in their environment. For example, with respect to electrical stimulation of nerve tissue in the brain, the reduction in efficacy due to neuroplasticity often occurs after just a few weeks of treatment. In order to regain the same efficacy, a new set of efficacious electrical stimulation parameters must be determined, the new set of parameters must be entered into the system, and the system is again used to electrically stimulate the nerve tissue according to the new set of parameters to continue to treat the condition. This results in the additional time and expense associated with a return visit to the treating physician for determining and entering the new set of parameters. Especially where treatment is to continue over a relatively long period of time, such as months or years, this additional time and expense poses a significant drawback.

### SUMMARY

In lieu of approaching neuroplasticity as an undesirable mechanism to be overcome, in accordance with some embodiments, neuroplasticity can be beneficially directed or promoted by neuromodulation stimulation in conjunction with application of neuromodulation stimulation to provide a therapeutic benefit to the patient. According to one representative embodiment, at least two sets of stimulation parameters are defined within an implantable neuromodulation system. One set of the stimulation parameters is selected to provide a therapeutic effect to a specific location within the patient's brain tissue via one or several electrodes positioned proximate to the location. For example, stimulation can be applied to damaged tissue in the brain to mitigate symptoms associated with the damage. Another set of stimulation parameters is selected for stimulation of a related second location of the patient's brain tissue via one or several electrodes positioned proximate to the second location. The second set of stimulation parameters promotes neuroplasticity. Specifically, the promoted neuroplasticity can encourage the second location to at least partially assume the function previously performed by the damaged tissue.

The foregoing has outlined rather broadly features and technical advantages in order that the detailed description that follows may be better understood. Additional features and advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. Novel features together with further objects and advantages will be better understood from the following description when considered in connection with the accompanying figures. It is to be expressly understood, however, that each of the figures is provided for the purpose of illustration and description only and is not intended as a-definition of the limits of the appended claims.

### BRIEF DESCRIPTION OF THE DRAWINGS

FIGS. 1A-1B illustrate example electrical stimulation systems providing modified neuroplasticity effects in a person's nerve tissue.

FIGS. 2A-2C illustrate example steps that may be used to implant an example electrical stimulation system into a person for electrical stimulation of target nerve tissue.

FIGS. 3A-31 illustrate example electrical stimulation leads that may be used to provide modified neuroplasticity effects in a person's nerve tissue.

FIG. 4 illustrates an example stimulation set.

FIG. 5 illustrates a number of example stimulation programs, each of which includes a number of stimulation sets.

FIG. 6 illustrates example execution of a sequence of stimulation sets within an example stimulation program.

### DETAILED DESCRIPTION

### I. DEFINITIONS

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

As used herein, the term “brain region” or “brain tissue” refers to any tissue comprising that part of the central nervous system. The brain stem tissue is also encompassed by the term brain region, including the diencephalon. The brain tissue can be defined based upon its anatomy and/or its function. Functionally, the brain can be defined as containing sensory systems (e.g., somatic (touch, pain and analgesia), visual, taste, auditory, smell, perception of motion, etc.); motor systems; and homeostasis and arousal systems (e.g., hypothalamus, limbic and cerebral cortex). Anatomically, the brain can be defined as telencephalon (e.g., cerebral hemispheres (e.g., frontal lobe, parietal lobe, temporal lobe, occipital lobe, insular lobe, limbic lobe, olfactory structures), basal ganglia, lateral ventricles, cerebral cortex, white matter); diencephalon (e.g., eptihalamus, thalamus, hypothalamus, subthalamus (e.g., subthalamic nuclei STN), third ventricle and associated structures); mesencephalon; pons; medulla oblongata; and cerebellum.

As used herein, the term “brain stem” refers to the stemlike part of the brain that connects the cerebral hemispheres with the spinal cord and comprises the medulla oblongata, the pons and the midbrain. The brain stem regulates both motor and sensory processes. Most of the cranial nerves are located in the brain stem. The cranial nerves may include, for example, olfactory nerve, optic, nerve, oculomoter nerve, trochlear nerve, trigeminal nerve, abducent nerve, facial nerve, vestibulocochlear nerve, glossopharyngeal nerve, vagal nerve, accessory nerve, and hypoglossal nerve. These cranial nerves perform three main functions: 1) they provide the motor and general sensory innervation of the skin, muscles, and joints in the head and neck; 2) they mediate vision, hearing, olfaction, and taste; and 3) they carry the parasympathetic innervation of autonomic ganglia that control visceral functions, e.g., breathing, heart rate, blood pressure, etc.

As used herein, the term “central neuronal tissue” refers to neuronal tissue associated with the brain, spinal cord or brain stem.

As used herein, the term “in communication” refers to one or more electrical stimulation leads and/or catheters being adjacent, in the general vicinity, in close proximity, or directly next to, or in direct contact or directly in the target tissue. Thus, one of skill in the art understands that the one or more electrical stimulation leads and/or catheters are “in communication” with the target tissue results in a modulation of neuronal activity associated with the target tissue. Still further, if the target tissue is brain tissue, “in communication” with brain tissue encompasses surrounding or adjacent white matter tracts or fibers leading to and from the brain tissue and/or white matter tracts or fibers that are contiguous with the brain tissue.

As used herein, the term “hypothalamus” is a region of the brain located below the thalamus, forming the major portion of the ventral region of the diencephalon and functioning to regulate certain metabolic processes and other autonomic activities. The hypothalamus links the nervous system to the endocrine system by synthesizing and secreting neurohormones often called releasing hormones because they function by stimulating the secretion of hormones from the anterior pituitary gland. The hypothalamus is also the area of the brain that controls body temperature, hunger and thirst, and circadian cycles.

As used herein the term “limbic system” encompasses the amygdala, hippocampus, septum, cingulate gyrus, cingulate cortex, hypothalamus, epithalamus, anterior thalamus, mammillary bodies, and fornix. The limbic system has connections throughout the brain, more particularly with the primary sensory cortices, including the rhinencephalon for smell, the autonomic nervous system via the hypothalamus, and memory areas. Yet further, the limbic system is involved in mood, emotion and thought.

As used herein, the term “motor system” refers to any axon, ganglion, nerve, fiber, nuclei, etc., that is involved in movement. The control of movement can be achieved at one of two levels: the spinal cord or the brain (e.g., descending spinal tracts, basal ganglia and the motor cortex and other association cortex (e.g., primary and secondary motor cortex)).

As used herein, the term “neuronal” refers to a neuron which is a morphologic and functional unit of the brain, spinal column, and peripheral nerves.

As used herein, the term “neurological disease” refers to conditions, disorders, and/or diseases that are associated with the nervous system. The nervous system comprises two components, the central nervous system, which is composed of the brain and the spinal cord, and the peripheral nervous system, which is composed of ganglia and the peripheral nerves that lie outside the brain and the spinal cord. One of skill in the art realizes that the nervous system may be separated anatomically, but functionally they are interconnected and interactive. Yet further, the peripheral nervous system is divided into the autonomic system (parasympathetic and sympathetic), the somatic system and the enteric system. Thus, any condition, disorder and/or disease that effects any component or aspect of the nervous system (either central or peripheral) is referred to as a neurological condition, disorder and/or disease. As used herein, the term “neurological” or “neurology” encompasses the terms “neuropsychiatric” or “neuropsychiatry” and “neuropsychological” or “neuropsychological”. Thus, a neurological disease, condition, or disorder includes, but is not limited to cognitive disorders, affective disorders, movement disorders, mental disorders, pain disorders, sleep disorders, etc. 0241 As used herein, the term “neuropsychiatry” or “neuropsychiatric” refers to conditions, disorders and/or diseases that relate to both organic and psychic disorders of the nervous system.

As used herein, the term “neuropsychological” or “neuropsychologic” refers to conditions, disorders and/or disease that relate to the functioning of the brain and the cognitive processors or behavior.

As used herein, the term “neuroplasticity” refers to the brain's ability to reorganize itself by forming new neural connections. Neuroplasticity allows the neurons (nerve cells) in the central nervous system to compensate for injury and disease and to adjust their activities in response to new situations or to changes in their environment.

As used herein, the term “peripheral neuronal tissue” refers to any neuronal tissue associated with a nerve root, root ganglion, or peripheral nerve that is outside the brain and the spinal cord.

As used herein, the term “peripheral nerve” refers a neuron or a bundle of neurons comprising a part of the peripheral nervous system. The nervous system comprises two general components, the central nervous system, which is composed of the brain and the spinal cord, and the peripheral nervous system, which is composed of ganglia or dorsal root ganglia and the peripheral nerves that lie outside the brain and the spinal cord. One of skill in the art realizes that the nervous system may be separated anatomically, but functionally they are interconnected and interactive. The peripheral nervous system is divided into the autonomic system (parasympathetic and sympathetic), the somatic system and the enteric system. The term peripheral nerve is intended to include both motor and sensory neurons and neuronal bundles of the autonomic system, the somatic system, and the enteric system that reside outside of the spinal cord and the brain. Peripheral nerve ganglia and nerves located outside of the brain and spinal cord are also described by the term peripheral nerve.

As used herein, the term “proximate” means on, in, adjacent, or near. Thus, one or more of the electrodes on an electrical stimulation lead are adapted to be positioned on, in, adjacent, or near target tissue.

As used herein, the term “sensory system” refers any axon, ganglion, nerve, fiber, nuclei, etc., that is involved in the coding and processing of sensation and perception. Thus, the sensory system consists of sensory receptors, neural pathways, and those parts of the brain responsible for processing the information. Sensory systems can include somatic sensation, vision, olfaction, taste and hearing. The thalamus plays an essential role in the sensory system in that the sensory information is processed by the thalamus and transmitted to the cerebral cortex.

As used herein, the term “somatosensory system” refers to the peripheral nervous system division comprising primarily afferent somatic sensory neurons and afferent visceral sensory neurons that receive sensory information from skin and deep tissue, including the 12 cranial and 21 spinal nerves. There are four distinct somatic modalities, including, touch, proprioceptive sensations, pain and thermal sensations.

As used herein, the term “somatosensory cortex” or “sensory cortex” includes the primary somatosensory cortex, secondary somatosensory cortex and the somatosensory association cortex, as well as the Brodmann areas associated therewith. Still further, the sensory cortex includes all cortical sites having projections to or from the sensory cortex, as well as the subcortical sites having projections to or from the sensory cortex. Primary somatosensory cortex refers to the brain region located in the postcentral gyrus and in the posterior part of the paracentral lobule. The primary somatosensory cortex also includes Brodmann areas 3, 1 and 2. The term secondary somatosensory cortex refers to the brain region that lies ventral to the primary somatosensory area along the superior bank of the lateral sulcus. The somatosensory association cortex refers to the brain areas of the superior parietal lobule, and supramarginal gyrus. The somatosensory association cortex also includes Brodmann areas 5, 7, and 40.

As used herein, “spinal cord,” “spinal nervous tissue associated with a vertebral segment,” “nervous tissue associated with a vertebral segment” or “spinal cord associated with a vertebral segment or level” includes any spinal nervous tissue associated with a vertebral level or segment. Those of skill in the art are aware that the spinal cord and tissue associated therewith are associated with cervical, thoracic and lumbar vertebrae. As used herein, C1 refers to cervical vertebral segment 1, C2 refers to cervical vertebral segment 2, and so on. T1 refers to thoracic vertebral segment 1, T2 refers to thoracic vertebral segment 2, and so on. L1 refers to lumbar vertebral segment 1, L2 refers to lumbar vertebral segment 2, and so on, unless otherwise specifically noted. In certain cases, spinal cord nerve roots leave the bony spine at a vertebral level different from the vertebral segment with which the root is associated. For example, the T11 nerve root leaves the spinal cord myelum at an area located behind vertebral body T8-T9 but leaves the bony spine between T11 and T12.

As used herein, the term “stimulate” or “stimulation” refers to electrical, stimulation that modulates at least the target tissue.

As used herein, the term “stimulation tissue” refers to the neuronal tissue that is stimulated to result in a desired therapeutic effect.

As used herein, the term “target tissue” refers to the neuronal tissue that has the neuroplastic change. The stimulation tissue can be tissue that would assume performance of the cognitive function previously performed by the target tissue if stimulated.

As used herein, the term “thalamus” refers to a part of the brain that is located in the center of the brain, beneath the cerebral hemispheres and next to the third ventricle. It is formed of grey matter and is considered to be relay station for nerve impulses in the brain.

As used herein, the term “treating” and “treatment” refers to modulating target neuronal sites (central neuronal tissue and/or peripheral neuronal tissue) so that the subject has an improvement in the disease or condition, for example, beneficial or desired clinical results. For purposes of this application, beneficial or desired clinical results include, but are not limited to, alleviation of symptoms, diminishment of extent of disease, stabilized (e.g., not worsening) state of disease, delay or slowing of disease progression, amelioration or palliation of the disease state, and remission (whether partial or total), whether detectable or undetectable. One of skill in the art realizes that a treatment may improve the disease condition, but may not be a complete cure for the disease.

### II. STIMULATION SYSTEM AND DEVICES

A: Mitigating or Reducing Neuroplasticity

As discussed in previously filed U.S. patent application Ser. No. 10/994,008, entitled “Electrical stimulation system, lead, and method of providing reduced neuroplasticity effects,” during the operation of a neurological stimulation system 10 according to a particular set of stimulation parameters, the efficacy of the stimulation associated with the particular set of stimulation parameters may be altered (e.g., decreased or increased) over time due to neuroplasticity. Neuroplasticity refers to the ability of the brain to dynamically reorganize itself in response to certain stimuli to form new neural connections. With respect to neuroplasticity of the spinal cord, stimuli may result in sprouting of dendrites into other areas. Still further, the peripheral nervous system may affect neuroplasticity in the central nervous system, stimulation of the peripheral nervous system feeds back to the central nervous system causing the central nervous system to reorganize itself in response. This allows the neurons in the central nervous system to compensate for injury or disease and adjust their activity in response to new situations or changes in their environment. With respect to electrical stimulation, an alteration in efficacy due to neuroplasticity can occur after a short period of time after treatment, for example, but not limited to just a few weeks of treatment. In order to regain the same efficacy, a new set of efficacious electrical stimulation parameters must be determined, the new set of parameters must be entered into the system, and the system is again used to electrically stimulate the neuronal tissue according to the new set of parameters to continue to treat the condition. This may result in the additional time and expense associated with a return visit to the treating physician for determining and entering the new set of parameters. Especially where treatment is to continue over a relatively long period of time, such as months or years, this additional time and expense poses a significant drawback. In other situations, such as when a patient has experienced a stroke, neuroplasticity effects may be desirable.

Some embodiments are designed to circumvent or reduce the need to reprogram new parameters. According to some embodiments, stimulation system 10 is used to provide electrical stimulation of target nerve tissue in a person's central nervous system (e.g., brain, spinal cord), or peripheral nervous system to reduce, enhance, or otherwise modify neuroplasticity effects. For example, the onset of neuroplasticity effects associated with therapeutic electrical stimulation of the nerve tissue may be prevented, delayed, or otherwise reduced. As a result, the efficacy period associated with a particular set of stimulation parameters may be extended. This may help prevent the additional time and expense associated with one or more return visits to the treating physician for determining and entering new sets of efficacious parameters. Especially where treatment is to continue over a relatively long period of time, such as months or years, avoiding this additional time and expense may provide a significant advantage. As another example, further development of neuroplasticity effects already in existence due to injury or disease may be prevented, delayed, or otherwise reduced, or such pre-existing neuroplasticity effects may be reversed in whole or in part.

The reduction or mitigation of neuroplasticity effects in the patient can be achieved by partially randomizing or otherwise varying the stimulation provided to the patient. According to some embodiments, an implantable neuromodulation system is employed to stimulate a first location according to a first set of stimulation parameters. The stimulation of the first location occurs to provide a therapeutic effect to a specific location with the patient's brain tissue via one or several electrodes positioned proximate to the location. By stimulating the patient through the first electrode(s) according to an appropriate set of stimulation parameters, the patient can experience a desired therapeutic effect (e.g., relief from tinnitus). In addition to stimulating the specific location, further stimulation is also applied to adjacent tissue in at least a somewhat distributed manner. The additional stimulation preferably occurs in a pseudo-randomized manner (e.g., in terms of electrode location, polarity, amplitude, pulse width, and/or frequency). It is believed that the diversity or randomization in the stimulation presents a lesser degree of stimulation “coherence” or “correlation” to which the neural tissue can adapt. Accordingly, neuroplasticity would likely progress at a slower rate (if at all). By reducing the rate of progression of neural adaptation, the effectiveness of the therapeutic stimulation is maintained for a longer period of time.

B. Directing or Promoting Neuroplasticity Effects

In certain other embodiments, stimulation system 10 is similarly capable of applying electrical stimulation to target nerve tissue to enhance, rather than reduce, neuroplasticity effects associated with the therapeutic electrical stimulation. For example, a patient may have experienced impairment of a cognitive function due to damage to a specific location with the patient's brain due to stroke, disease, physical injury, etc. System 10 can be programmed to stimulate the damaged location to provide a therapeutic benefit (e.g., to treat symptoms associated with the damage). In addition to stimulating the damaged location, system 10 can also be programmed to stimulate other adjacent tissue. The purpose of stimulating the other tissue is to promote neuroplasticity effects to encourage the other location to assume performance of the impaired neuronal function. Specifically, it is believed that stimulation of normally functioning neural tissue adjacent to damaged neural tissue advances the rate at which neuroplasticity effects allow the recovery of impaired neuronal functions.

C. Implantable Generator, Lead, and Methods of Stimulations

Whether stimulation is applied for the purpose of mitigating neuroplasticity effects or enhancing neuroplasticity effects, the same types of implantable generator and leads devices can be employed. Specifically, the implementation difference between the treatment options is achieved by appropriately positioning the lead electrodes relative to the corresponding neural tissue and by programming the implantable generator to deliver suitable pulses to the corresponding neural tissue.

The target tissue and/or stimulation tissue is neuronal tissue which includes any tissue associated with the peripheral nervous system or the central nervous system. Peripheral neuronal tissue can include a nerve root or root ganglion or any neuronal tissue that lies outside the brain, brainstem or spinal cord. Peripheral nerves can include, but are not limited to olfactory nerve, optic, nerve, oculomotor nerve, trochlear nerve, trigeminal nerve, abducens nerve, facial nerve, vestibulocochlear (auditory) nerve, glossopharyngeal nerve, vagal nerve, accessory nerve, hypoglossal nerve, suboccipital nerve, the greater occipital nerve, the lesser occipital nerve, the greater auricular nerve, the lesser auricular nerve, the phrenic nerve, brachial plexus, radial axillary nerves, musculocutaneous nerves, radial nerves, ulnar nerves, median nerves, intercostal nerves, lumbosacral plexus, sciatic nerves, common peroneal nerve, tibial nerves, sural nerves, femoral nerves, gluteal nerves, thoracic spinal nerves, obturator nerves, digital nerves, pudendal nerves, plantar nerves, saphenous nerves, illoinguinal nerves, gentofemoral nerves, and iliohypogastric nerves.

Central neuronal tissue includes brain tissue, spinal tissue or brainstem tissue. Brain tissue can include thalamus/sub-thalamus (e.g., subthalamic nuclei (STN)), basal ganglia, hippocampus, amygdala, hypothalamus, mammilary bodies, cingulate gyrus, subcingulate gyrus, substantia nigra or cortex (e.g., primary and/or secondary somatosensory cortex or primary and/or secondary motor cortex) or white matter tracts afferent to or efferent from the abovementioned brain tissue, inclusive of the corpus callosum. Spinal tissue can include the ascending and descending tracts of the spinal cord. The brainstem tissue can include the medulla oblongata, pons or mesencephalon. Brain tissue can also include any tissue (white matter and/or gray matter) related to the functional systems of the brain, for example, but not limited to sensory systems (e.g., somatic (touch, pain and analgesia), visual, taste, auditory, smell, perception of motion, etc.); motor systems; and homeostasis and arousal systems (e.g., hypothalamus, limbic and cerebral cortex). Still further, other brain tissue target areas can include any of the known Broadmann Areas, for example, but not limited to Broadmann Area 1, 2, 3, 4, 5, 6, 7, 8, 9, 11, 12, 13, 17, 19, 20, 22, 23, 24, 25, 28, 29, 30, 33, 38 and 46.

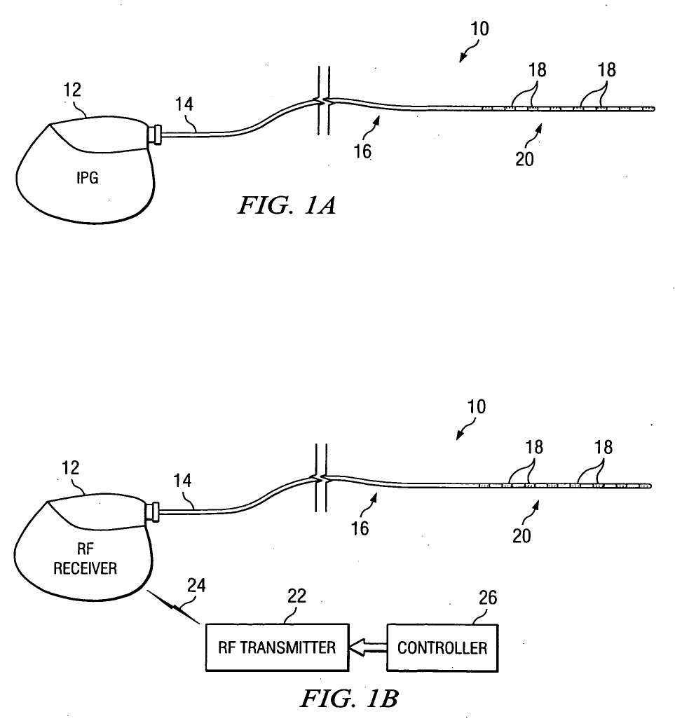

FIGS. 1A-1B illustrate example electrical stimulation systems 10 used to provide reduced, enhanced, or otherwise modified neuroplasticity effects associated with therapeutic electrical stimulation of the brain, the spinal cord, or a peripheral or other nerve or due to previous injury or disease of the brain, the spinal cord, or a peripheral or other nerve. Stimulation system 10 generates and applies a stimulus to a target area of the brain, the spinal cord, or a peripheral or other nerve. In general terms, stimulation system 10 includes an implantable electrical stimulation source 12 and an implantable electrical stimulation lead 14 for applying the electrical stimulation pulses to target nerve tissue. In operation, both of these primary components are implanted in the person's body. Stimulation source 12 is coupled to a connecting portion 16 of stimulation lead 14. In certain other embodiments, stimulation source 12 and electrodes are contained in an “all-in-one” microstimulator or other unit, such as a Bion® microstimulator manufactured by Advanced Bionics Corporation. In any case, the stimulation source 12 controls the electrical stimulation pulses transmitted to electrodes located on a stimulating portion 20 of stimulation lead 14, which is positioned on, in, near, or otherwise proximate the target nerve tissue, according to suitable stimulation parameters (e.g., duration, amplitude or intensity information, frequency information, etc.). A doctor, the patient, or another user of stimulation source 12 may directly or indirectly input stimulation parameters to specify or modify the electrical stimulation provided.

In one embodiment, as shown in FIG. 1A, stimulation source 12 includes an implantable pulse generator (IPG). An example IPG may be one manufactured by Advanced Neuromodulation Systems, Inc., such as the Genesis® System, part numbers 3604, 3608, 3609, and 3644. In another embodiment, as shown in FIG. 1B, stimulation source 12 includes an implantable wireless receiver. Examples of an IPG including wireless functionality and adapted for deep brain stimulation are the Libra™ DBS IPGs (Model No.s 6608 and 6644) manufactured by Advanced Neuromodulation Systems, Inc. Additionally, the Libra™ DBS IPGs include multi-program and multi-stimulation set capability. Such capability may be employed to stimulate damaged tissue and other related tissue to reduce neuroplasticity or promote neuroplasticity depending upon the programming of the stimulation sets and/or stimulation programs. The wireless receiver is capable of receiving wireless signals from a wireless transmitter 22 located external to the person's body. The wireless signals are represented in FIG. 1B by wireless link symbol 24. A doctor, the patient, or another user of stimulation source 12 may use a controller 26 located external to the person's body to provide control signals for operation of stimulation source 12. Controller 26 provides the control signals to wireless transmitter 22, wireless transmitter 22 transmits the control signals and power to the wireless receiver of stimulation source 12, and stimulation source 12 uses the control signals to vary the stimulation parameters of electrical stimulation pulses transmitted through stimulation lead 14 to the stimulation site. An example wireless transmitter 22 may be one manufactured by Advanced Neuromodulation Systems, Inc., such as the Clinician Programmer device. Wireless transmitter 22 can be used as a programming device to set the programmable operational parameters of stimulation source 12. Wireless transmitter 22 can also be optionally operated in conjunction with programming software executed on a computer system (not shown). Stimulation source 12 can be programmed by wireless transmitter 22 by identifying an electrode or electrodes that are adjacent to damaged tissue after implantation of a suitable stimulation lead. The clinician can then construct a first set of operational parameters (pulse amplitude, pulse width, frequency, electrode polarity, etc.) for stimulation of the damaged tissue via the identified electrodes that provide a therapeutic benefit. The first set of operational parameters can be downloaded to stimulation source 12 to control its operations. Also, another electrode or electrodes are selected that are adjacent to tissue to be stimulated for the purpose of promoting neuroplasticity. A second set of operational parameters are downloaded to stimulation source 12 for stimulation via the second electrode(s) in a manner that promotes neuroplasticity and does not otherwise impair the function of the tissue. Some parameters of the second set of operational parameters may be predefined and simply selected by the clinician for downloading to stimulation source 12. Additionally, the operational parameters can be downloaded to a patient controller device. After the downloading operations are completed, the IPG can be activated to stimulate both locations according to the downloaded parameters.

### III. IMPLANTATION OF DEVICES

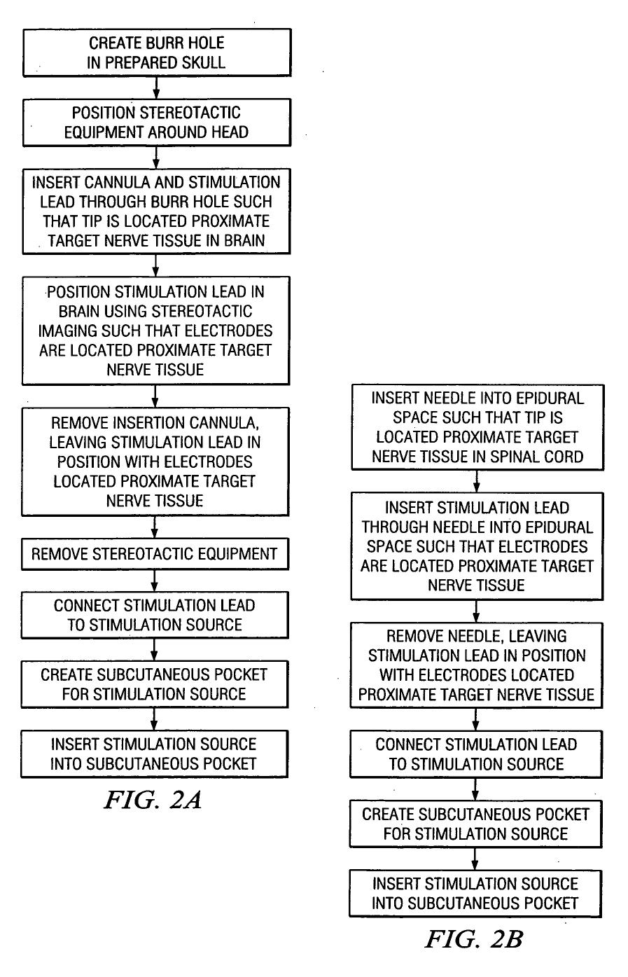

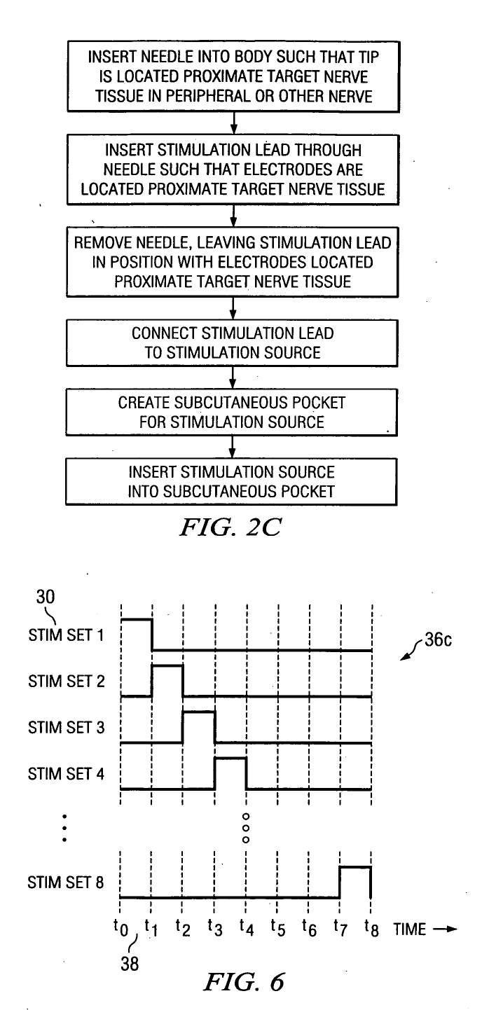

FIGS. 2A-2C illustrate example steps that may be used to implant an example stimulation system 10 into a person for electrical stimulation of the person's nerve tissue, for example, electrical stimulation of target nerve tissue in the brain, the spinal cord, or a peripheral or other nerve.

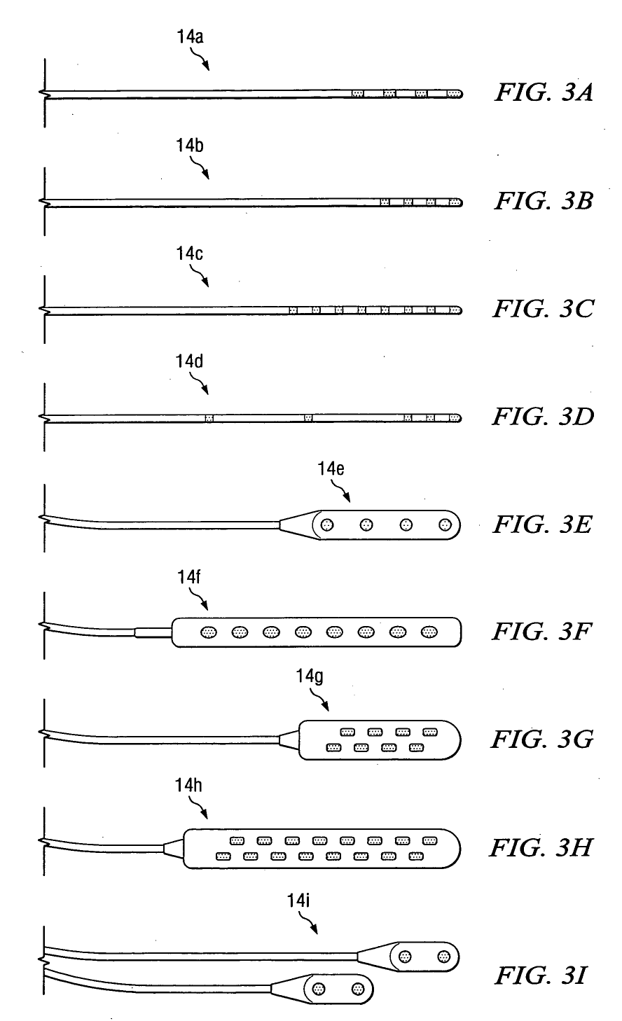

FIGS. 3A-31 illustrate example stimulation leads 14 that may be used to provide reduced, enhanced, or otherwise modified neuroplasticity effects in a person's brain, for example, associated with therapeutic electrical stimulation of the brain or due to previous injury or disease. Each of the one or more stimulation leads 14 incorporated in stimulation system 10 includes one or more electrodes adapted to be positioned on, in, near, or otherwise proximate the target nerve tissue and used to deliver to the target nerve tissue electrical stimulation pulses received from stimulation source 12. A percutaneous stimulation lead 14, such as example stimulation leads 14a-d, includes one or more circumferential electrodes spaced apart from one another along the length of stimulation lead 14. Circumferential electrodes emit electrical stimulation energy generally radially in all directions. A laminotomy or paddle style stimulation lead 14, such as example stimulation leads 14e-i, includes one or more directional electrodes spaced apart from one another along one surface of stimulation lead 14. Directional electrodes emit electrical stimulation energy in a direction generally perpendicular to the surface of stimulation lead 14 on which they are located. Although various types of stimulation leads 14 are shown as examples, stimulation system 10 may employ any suitable type of stimulation lead 14 in any suitable number.

An example of an eight-electrode percutaneous lead is an OCTRODE® lead manufactured by Advanced Neuromodulation Systems, Inc. A stimulation system such as is described in U.S. Pat. No. 6,748,276 is also contemplated. Circumferential electrodes emit electrical stimulation energy generally radially in all directions.

A laminotomy, paddle, or surgical stimulation lead 14, such as example stimulation leads 14 described in FIGS. 3E-I, includes one or more directional stimulation electrodes spaced apart from one another along one surface of stimulation lead 14. An example of an eight-electrode, two column laminotomy lead is a LAMITRODE® and C-series LAMITRODE® 44 leads manufactured by Advanced Neuromodulation Systems, Inc. Directional stimulation electrodes emit electrical stimulation energy in a direction generally perpendicular to the surface of stimulation lead 14 on which they are located.

Referring to FIG. 2A, in one embodiment, for stimulation of a person's brain, the skull is first prepared by exposing the skull and creating a burr hole in the skull. A burr hole cover may be seated within the burr hole and fixed to the scalp or skull. Stereotactic equipment suitable to aid in placement of an electrical stimulation lead 14 in the brain may be positioned around the head. Typically, an insertion cannula for stimulation lead 14 is inserted through the burr hole into the brain, but a cannula is not required. For example, a hollow needle may provide the cannula. The cannula and stimulation lead 14 may be inserted together or stimulation lead 14 may be inserted through the cannula after the cannula has been inserted. Using stereotactic imaging guidance or otherwise, stimulation lead 14 precisely positioned in the brain such that electrodes are precisely positioned in, on, near, or otherwise proximate the target nerve tissue.

Brain tissue can include thalamus/sub-thalamus, subthalmalic nuclei, basal ganglia, hippocampus, amygdala, hypothalamus, mammilary bodies, cingulate gyrus, subcingulate gyrus, substantia nigra or cortex (primary and/or secondary somatosensory cortex or primary and/or secondary motor cortex) or white matter tracts afferent to or efferent from the abovementioned brain tissue, inclusive of the corpus callosum. Brain tissue can also include any tissue (white matter and/or gray matter) related to the functional systems of the brain, for example, but not limited to sensory systems (e.g., somatic (touch, pain and analgesia), visual, taste, auditory, smell, perception of motion, etc.); motor systems; and homeostasis and arousal systems (e.g., hypothalamus, limbic and cerebral cortex).

In certain embodiments, the target tissue and/or stimulation tissue can be selected from tissues associated with the limbic system and/or the somatosensory system, more specifically, the somatosensory cortex and/or the motor cortex. The limbic system includes, but is not limited to encompasses the amygdala, hippocampus, septum, cingulate gyrus, subcingulate gyrus, cingulate cortex, hypothalamus, epithalamus, anterior thalamus, mammillary bodies, and fornix. The limbic system has connections throughout the brain, more particularly with the primary sensory cortices, including the rhinencephalon for smell, the autonomic nervous system via the hypothalamus, and memory areas. The term somatosensory cortex includes, but is not limited to the primary somatosensory cortex, secondary somatosensory cortex and the somatosensory association cortex, as well as the Brodmann areas associated therewith, as well as all cortical sites having projections to or from the sensory cortex, as well as the subcortical sites having projections to or from the sensory cortex. The motor cortex includes, but is not limited to the primary motor cortex, the premotor cortex, the supplementary motor cortex and the frontal eye field, as well as the Brodmann areas associated therewith, and any cortical sites having projections to or from the motor cortex and any subcortical sites having projections to or from the motor cortex.

Other brain tissue areas can include, but are not limited primary visual cortex, secondary and tertiary visual cortices, visual association cortex, primary auditory cortex, auditory association cortex, gustatory cortex, and vestibular cortex, other brain regions that receive somatic inputs, for example, the posterior parietal lobe, as well as any brain region that is stimulated by sensory stimulation, such as the cerebellum. Other areas of higher cortical function can include, prefrontal cortex, Broca's speech area, Wernicke's speech area, Arcuate fasciculus, and the corpus callosum.

Once stimulation lead 14 has been positioned in the brain, stimulation lead 14 is uncoupled from any stereotactic equipment and the cannula and stereotactic equipment are removed. Where stereotactic equipment is used, the cannula may be removed before, during, or after removal of the stereotactic equipment. Connecting portion 16 of stimulation lead 14 is laid substantially flat along the skull. Where appropriate, any burr hole cover seated in the burr hole may be used to secure stimulation lead 14 in position and possibly to help prevent leakage from the burr hole and entry of contaminants into the burr hole. Example burr hole covers that may be appropriate in certain embodiments are illustrated and described in copending U.S. Provisional Application Nos. 60/528,604 and 60/528,689, both filed Dec. 11, 2003 and entitled “Electrical Stimulation System and Associated Apparatus for Securing an Electrical Stimulation Lead in Position in a Person's Brain” (Attorney's Docket 065274.0113 and 065274.0120).

In some embodiments, stimulation of the spinal cord may occur in addition to direct stimulation of brain tissue for the purpose of altering neuroplasticity within brain tissue of the patient. Yet further, stimulation of the spinal cord may result in alterations of neuroplasticity within the stimulation site or surrounding tissue of the spinal cord. Referring to FIG. 2B, in another embodiment, for stimulation of a person's spinal cord using a percutaneous stimulation lead 14, a needle may be inserted into the epidural space such that the tip of the needle is located in, on, near, or otherwise proximate target nerve tissue in the spinal cord. Stimulation lead 14 may be inserted through the needle into the epidural space such that electrodes of stimulation lead 14 are located in, on, near, or otherwise proximate the target nerve tissue in the spinal cord. The needle may then be removed leaving stimulation lead 14 in position with electrodes located proximate the target nerve tissue. As an alternative example, after the needle is inserted, a guide wire may be inserted through the needle into position, the needle may be removed, an introducer may be advanced along the guide wire into position, stimulation lead 14 may be inserted through the introducer into position, and the introducer may then be removed leaving stimulation lead 14 in position with electrodes again located proximate the target nerve tissue. In other embodiments, a laminotomy or paddle style stimulation lead 14 may be implanted using a surgical or other technique.

Electrical energy can be delivered through electrodes positioned external to the dura layer surrounding the spinal cord. Stimulation on the surface of the cord (subdurally) is also contemplated, for example, stimulation may be applied to the dorsal columns as well as to the dorsal root entry zone or the dorsal root ganglia and/or nerve root. Any area of the spinal cord may be stimulated for example the any neuronal tissue associated with any of the cervical vertebral segments (C1, C2, C3, C4, C5, C6, C7 and C8) and/or any tissue associated with any of the thoracic vertebral segments (T1, T2, T3, T4, T5, T6, T7, T8, T9, T10, T11, 12) and/or any tissue associated with any of the lumbar vertebral segments (L1, L2, L3, L4, L5, L6) and/or any tissue associated with the sacral vertebral segments (S1, S2, S3, S4, S5).

In some embodiments, stimulation of one or several peripheral nerves may occur in addition to direct stimulation of brain tissue for the purpose of altering neuroplasticity within brain tissue of the patient. Referring to FIG. 2C, in another embodiment, for stimulation of a person's peripheral or other nerve using a percutaneous stimulation lead 14, a needle may be inserted into the body such that the tip of the needle is located in, on, near, or otherwise proximate target nerve tissue in the peripheral or other nerve. Stimulation lead 14 may be inserted through the needle into the body such that electrodes of stimulation lead 14 are located in, on, near, or otherwise proximate the target nerve tissue in the peripheral or other nerve. The needle may then be removed leaving stimulation lead 14 in position with electrodes located proximate the target nerve tissue. As an alternative example, after the needle is inserted, a guide wire may be inserted through the needle into position, the needle may be removed, an introducer may be advanced along the guide wire into position, stimulation lead 14 may be inserted through the introducer into position, and the introducer may then be removed leaving stimulation lead 14 in position with electrodes again located proximate the target nerve tissue. In other embodiments, a laminotomy or paddle style stimulation lead 14 may be implanted using a surgical or other technique.

Although various types of stimulation leads 14 are shown as examples, the stimulation system 10 may employ any suitable type of stimulation lead 14 in any suitable number. In addition, stimulation leads 14 may be used alone or in combination. For example, medial or unilateral stimulation of the predetermined site may be accomplished using a single electrical stimulation lead 14 implanted in communication with the predetermined site in one side of the head, while bilateral electrical stimulation of the predetermined site may be accomplished using two stimulation leads 14 implanted in communication with the predetermined site in opposite sides of the body.

Whether using percutaneous leads, laminotomy leads, or some combination of both, the leads are coupled to one or more neurostimulation devices, or signal generators. The devices can be totally implanted systems and/or radio frequency (RF) systems. An example of an RF system is a MNT/MNR-916CC system manufactured by Advanced Neuromodulation Systems, Inc.

Still further, a contemplated stimulation system may have no leads, with the electrodes directly connected to the pulse generator. Alternatively, in another embodiment, a stimulation system with flexible leads is also contemplated. One with skill in the art realizes that neuroplasticity effecting methods are appropriate for use with a wide range of stimulation devices capable of providing stimulation to peripheral nervous tissue.

In one embodiment, the stimulation source is transcutaneously in communication with the electrical stimulation lead. In “transcutaneous”, electrical nerve stimulation (TENS) the stimulation source is external to the patient's body, and may be worn in an appropriate fanny pack or belt, and the electrical stimulation lead is in communication with the stimulation source, either remotely or directly. In another embodiment, the stimulation is percutaneous. In “percutaneous” electrical nerve stimulation (PENS), needles are inserted to an appropriate depth around or immediately adjacent to a predetermined stimulation site, and then stimulated.

Peripheral nerves can include, but are not limited to olfactory nerve, optic, nerve, oculomotor nerve, trochlear nerve, trigeminal nerve, abducens nerve, facial nerve, vestibulocochlear (auditory) nerve, glossopharyngeal nerve, vagal nerve, accessory nerve, hypoglossal nerve, suboccipital nerve, the greater occipital nerve, the lesser occipital nerve, the greater auricular nerve, the lesser auricular nerve, the phrenic nerve, brachial plexus, radial axillary nerves, musculocutaneous nerves, radial nerves, ulnar nerves, median nerves, intercostal nerves, lumbosacral plexus, sciatic nerves, common peroneal nerve, tibial nerves, sural nerves, femoral nerves, gluteal nerves, thoracic spinal nerves, obturator nerves, digital nerves, pudendal nerves, plantar nerves, saphenous nerves, ilioinguinal nerves, gentofemoral nerves, and iliohypogastric nerves.

Once stimulation lead 14 has been inserted and secured, connecting portion 16 of stimulation lead 14 extends from the lead insertion site to the implant site at which stimulation source 12 is implanted. The implant site is typically a subcutaneous pocket formed to receive and house stimulation source 12. The implant site is usually some distance away from the insertion site, such as in or near the chest or buttocks. A doctor, the patient, or another user of stimulation source 12 may directly or indirectly input stimulation parameters for controlling the nature of the electrical stimulation provided.

Yet in further embodiments, the stimulation system 10, described above, can be implanted into a person's body with stimulation lead 14 located in communication with a target brain stem tissue and/or area. Such systems that can be used are described in WO2004062470, which is incorporated herein by reference in its entirety.

The predetermined brain stem tissue can be selected from medulla oblongata, pons or mesencephalon.

Implantation of a stimulation lead 14 in communication with the target brain stem area can be accomplished via a variety of surgical techniques that are well known to those of skill in the art. For example, an electrical stimulation lead can be implanted on, in, or near the brain stem by accessing the brain tissue through a percutaneous route, an open craniotomy, or a burr hole. Where a burr hole is the means of accessing the brainstem, for example, stereotactic equipment suitable to aid in placement of an electrical stimulation lead 14 on, in, or near the brain stem may be positioned around the head. Another alternative technique can include, a modified midline or retrosigmoid posterior fossa technique.

In certain embodiments, electrical stimulation lead 14 is located at least partially within or below the aura mater adjacent the brainstem. Alternatively, a stimulation lead 14 can be placed in communication with the predetermined brainstem area by threading the stimulation lead up the spinal cord column, as described above, which is incorporated herein.

As described above, each of the one or more leads 14 incorporated in stimulation system 10 includes one or more electrodes adapted to be positioned near the target brain tissue and used to deliver electrical stimulation energy to the target brain tissue in response to electrical signals received from stimulation source 12. A percutaneous lead 14 may include one or more circumferential electrodes spaced apart from one another along the length of lead 14. Circumferential electrodes emit electrical stimulation energy generally radially in all directions and may be inserted percutaneously or through a needle. The electrodes of a percutaneous lead 14 may be arranged in configurations other than circumferentially, for example as in a “coated” lead 14. A laminotomy or paddle style lead 14, such as example leads 14e-i, includes one or more directional electrodes spaced apart from one another along one surface of lead 14. Directional electrodes emit electrical stimulation energy in a direction generally perpendicular to the surface of lead 14 on which they are located. Although various types of leads 14 are shown as examples, any suitable type of lead 14 in any suitable number, including three-dimensional leads and matrix leads as described below may be employed. In addition, the leads may be used alone or in combination.

Yet further, a stimulation lead 14 can be implanted in communication with the target brain stem area by a using stereotactic procedures similar to those described above, which are incorporated herein, for implantation via the cerebrum. 072 Still further, a target brain stem area can be indirectly stimulated by implanting a stimulation lead 14 in communication with a cranial nerve (e.g., olfactory nerve, optic, nerve, oculomoter nerve, trochlear nerve, trigeminal nerve, abducent nerve, facial nerve, vestibulocochlear nerve, glossopharyngeal nerve, vagal nerve, accessory nerve, and the hypoglossal nerve) such that stimulation of a cranial nerve indirectly stimulates the predetermined brainstem tissue. Such techniques are further described in U.S. Pat. Nos. 6,721,603; 6,622,047; and 5,335,657, and U.S. Provisional Application 60/591,195 entitled “Stimulation System and Method for Treating a Neurological Disorder” each of which are incorporated herein by reference.

Although example steps are illustrated and described, two or more steps may be performed or implemented to take place substantially simultaneously or in a different order. In addition, neuroplasticity effecting methods can be employed or implemented with additional steps, fewer steps, or different steps, so long as the steps remain appropriate for implanting an example stimulation system 10 into a person for electrical stimulation of target nerve tissue in the person's brain, spinal cord, or peripheral or other nerve.

### IV. Modulating Neuroplasticity

The nervous system comprises two general components, the central nervous system, which is composed of the brain and the spinal cord, and the peripheral nervous system, which is composed of ganglia or dorsal root ganglia and the peripheral nerves that lie outside the brain and the spinal cord. One of skill in the art realizes that the nervous system may be linguistically separated and categorized, but functionally they are interconnected and interactive.

The central nervous system comprises the brain and spinal cord, which together function as the principal integrator of sensory input and motor output. In general terms, the brain consists of the cerebrum (cerebral hemispheres and the diencephalons), the brainstem (midbrain, pons, and medulla); and the cerebellum. It is well known that the cerebrum represents the highest center for sensory and motor processing. In general, the frontal lobe processes motor, visual, speech, and personality modalities; the parietal lobe processes sensory information; the temporal lobe, auditory and memory modalities; and the occipital lobe vision. The cerebellum, in general, coordinates smooth motor activities and processes muscle position, while the brainstem conveys motor and sensory information and mediates important autonomic functions. These structures are of course integrated with the spinal cord which receives sensory input from the body and conveys somatic and autonomic motor information to peripheral targets. Thus, one of skill in the art realizes that the central nervous system is capable of evaluating incoming information and formulating response to changes that threaten the homeostasis of the individual.

The peripheral nervous system is divided into the autonomic system (parasympathetic and sympathetic), the somatic system and the enteric system. The term peripheral nerve is intended to include both motor and sensory neurons and neuronal bundles of the autonomic system, the somatic system, and the enteric system that reside outside of the spinal cord and the brain. Peripheral nerve ganglia and nerves located outside of the brain and spinal cord are also described by the term peripheral nerve.

Neuroplasticity refers to the ability of the brain to dynamically reorganize itself in response to certain stimuli to form new neural connections. With respect to neuroplasticity of the spinal cord, stimuli may result in sprouting of dendrites into other areas. Still further, the peripheral nervous system may affect neuroplasticity in the central nervous system, stimulation of the peripheral nervous system feeds back to the central nervous system causing the central nervous system to reorganize itself in response. This allows the neurons in the central nervous system to compensate for injury or disease and adjust their activity in response to new situations or changes in their environment. With respect to electrical stimulation, an alteration in efficacy due to neuroplasticity can occur after a short period of time after treatment, for example, but not limited to just a few weeks of treatment.

In some embodiments, a stimulation system 10 can be programmed to maintain the efficacy of therapeutic electrical stimulation. The stimulation system 10 is used to provide electrical stimulation of target nerve tissue in a person's central nervous system (e.g., brain, spinal cord), or peripheral nervous system to reduce, enhance, or otherwise modify neuroplasticity effects. For example, the onset of neuroplasticity effects associated with therapeutic electrical stimulation of the nerve tissue may be prevented, delayed, or otherwise reduced. As a result, the efficacy period associated with a particular set of stimulation parameters may be extended. This may help prevent the additional time and expense associated with one or more return visits to the treating physician for determining and entering new sets of efficacious parameters. Especially where treatment is to continue over a relatively long period of time, such as months or years, avoiding this additional time and expense may provide a significant advantage. As another example, further development of neuroplasticity effects already in existence due to injury or disease may be prevented, delayed, or otherwise reduced, or such pre-existing neuroplasticity effects may be reversed in whole or in part. The mitigation of neuroplasticity effects can occur by providing a varied and/or pseudo-randomized electrical stimulation by suitably controlling the programmable parameters of stimulation system 10.

Other embodiments stimulate brain tissue for the purpose of promoting neuroplasticity effects. For example, when a neuronal function (e.g., cognitive, motor, behavioral, etc.) is impaired due to damage of specific tissue in the brain or spinal cord, neuromodulation stimulation can be applied to that specific tissue to provide a therapeutic benefit associated with the impaired neuronal function. In addition to stimulating the damaged tissue, further stimulation can be applied to related brain tissue and/or spinal cord for the purpose of causing neuroplasticity. Specifically, promoted neuroplasticity can encourage the related tissue to at least partially assume performance of the neuronal function.

Impairment of neuronal function can be a result of stroke, disease, and/or physical injury (e.g., traumatic brain injury (TBI), etc.). Disease can be neurological disorders for example, but not limited to Developmental Disabilities [e.g., Cerebral Palsy, Mental Retardation, Attention Deficit Disorder (ADD), Pervasive Developmental Disorders and Autistic Spectrum Disorders (e.g., autism and Asperger's disorder), Learning Disabilities (e.g., dyslexa, disorders of motor functions (e.g., dysgraphia, dyspraxia, clumsiness), and nonverbal learning disabilities (e.g., dyscalculia, visuospatial dysfunction, socioemotional disabilities, and ADHD)]; Demyleinating Diseases [e.g., Multiple Sclerosis]; delirium and dementia [e.g., vascular dementia, dementia due to Parkinson's disease, dementia due to HIV disease, dementia due to Huntington's disease, and dementia due to Creutzfeld-Jakob disease; Alzheimer's dementia, multi-infarct dementia, stroke]; affective disorder [e.g., depression, mania, mood disorder, major depressive disorder, bipolar]; movement disorders [e.g, restless leg syndrome, Dyskinesia (e.g., tremor, dystonia, chorea and ballism, tic syndromes (e.g., Tourette's Syndrome), myoclonus, drug-induced movement disorders, Wilson's Disease, Paroxysmal Dyskinesias, Stiff Man Syndrome) and Akinetic-Ridgid Syndromes and Parkinsonism]; ataxic disorders [e.g., disturbances of gait]; substance abuse related disorders [e.g., alcohol use disorders, amphetamine use disorders, cannabis use disorders, caffeine induced disorders, cocaine use disorders, inhalant use disorders, opioid use disorders, hallucinogen disorders, sedative, hypnotic, or anxiolytic use disorders, and polysubstance use disorders]; sexual dysfunctions [e.g., sexual arousal disorder, male erectile disorder, female dyspareunia, male hypoactive disorder, and female hypoactive disorder]; eating disorders [e.g., overeating disorder, bulimia nervosa, and anorexia nervosa]; obesity, anxiety and obsessive compulsive disorder syndromes [e.g., anxiety, panic attacks, post-traumatic stress disorder, agoraphobia, obsessive and compulsive behavior]; impulse control disorders [e.g., pathological gambling, intermittent explosive disorder, kleptomania, and pyromania]; personality disorders (e.g., schizoid personality disorder, paranoid personality disorder, schizotypal personality disorder, borderline personality disorder, narcissistic personality disorder, histrionic personality disorder, obsessive compulsive personality disorder, avoidant personality disorder, dependent personality disorder, and anti-social personality disorder); and other psychiatric disorders [e.g., schizophrenia subtypes, schizoaffective disorder, schizophrenia undifferentiated, delusional disorder, cyclothymic disorder, somatoform disorder, hypochondriasis, dissociative disorder, and depersonalization disorder]; and Chiari I malformation.

The disease may also be related to gastric motility conditions. The gastrointestinal disorders or conditions contemplated by the present application include gastrointestinal altered motility, sensitivity and secretion disorders in which one or more of the symptoms and conditions affect the gastrointestinal tract from the mouth to the anus. Gastrointestinal disorders include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, gastroesophageal reflux disease, irritable bowel syndrome, ulcerative colitis, Crohn's disease, menstrual cramps, pancreatitis, spastic and interstitial cystitis and ulcers and the visceral pain associated therewith.

Other diseases may include immune-diseases, for example, but riot limited to arthritis (e.g., rheumatoid arthritis and psoriatic arthritis), inflammatory bowel diseases (e.g., ulcerative colitis and Crohn's disease), endocrinopathies (e.g., type 1 diabetes and Graves disease), neurodegenerative diseases (e.g., multiple sclerosis, autistic spectrum disorder, Alzheimer's disease, Guillain-Barre syndrome, obsessive-compulsive disorder, optic neuritis, retinal degeneration, amyotrophic lateral sclerosis (ALS), Parkinson's disease, Huntington's Disease, Guillain-Barre syndrome, myasthenia gravis, and chronic idiopathic demyelinating disease (CID)), vascular diseases (e.g., autoimmune hearing loss, systemic vasculitis, and atherosclerosis), and skin diseases (e.g., dermatomyositis, systemic lupus erthematosus, discoid lupus erthematosus, scleroderma, and vasculitics). More specifically, the immune-mediated include, but are not limited to rheumatoid arthritis, eryematos, Sojourn's syndrome, allergic asthma, atopic skin disease, chronic fatigue syndrome, allergies, and Chron's disease.

Still further, the disease may be a neuroendocrine disorder which relates to a disorder associated with the crosstalk that occurs between the endocrine system and the nervous system. More particularly, the disorders are associated with the hypothalamic-pituitary-adrenal (HPA) and -gonadal (HPG) axes, as well as disorders associated with the autonomic nervous system. Diseases associated with the HPA axis can include, but are not limited to pituitary tumors, Cushing syndrome, adrenal insufficiency, ACTH resistance, Congenital Adrenal Hyperplasia (CAH), adrenocortical tumors, glucocorticoid resistance/hypersensitivity, and mineralocorticoid resistance. Diseases of the HPG axis can include, but are not limited to hypothalamic hypogonadism, disturbances of the menstrual cycle, ovarian and testicular gonadotropin resistance, endometriosis, and infertility. Disease associated with the autonomic nervous system can include, but are not limited to pheochromocytoma and catecholamine deficiency. Still further, developmental/psychiatric, metabolic and immune disorders related to the functions of the HPA and HPG axes and the autonomic system can include, but are not limited to premature adrenarche, eating disorders—including anorexia and bulimia nervosa and adolescent obesity-, adolescent conduct disorder, dysthymia and depression, childhood asthma and rheumatoid arthritis, the premenstrual tension syndrome, and postpartum and climacteric depression and autoimmunity.

Other diseases may include cardiovascular disorders. Thus, the present application may enhance or improve cardiac function, for example, hemodynamics, electrical activity, myocontractility, perfusion of the heart muscle, as well as enhance cardiac performance or efficiency, such as balance between supply and demand. Cardiovascular disorders can include but are not limited to diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). Other symptoms that can be related cardiovascular diseases include cholesterol and/or blood pressure. Thus, the present application can be used to decrease cholesterol levels and/or to regulate blood pressure, for example decrease blood pressure in a subject suffering from high blood pressure.

In certain embodiments, the cortex may be the target site to be stimulated. The cortex of a person's brain functions to provide a person with a representation of the external environment to allow the person to function effectively in that environment. The cortex includes frontal, parietal, occipital, and temporal regions that are each generally associated with particular functions.

The frontal cortex is generally associated with control of motor abilities and includes what is commonly referred to as the primary motor cortex. The frontal cortex also includes a region referred to as the prefrontal cortex that receives sensory information of multiple types, including autonomic sensory information from the internal organs, and is considered important for guiding behavior based on memory, translating ideas into words, and other functions. The parietal cortex is generally associated with sensory perception of the external environment and includes what is commonly referred to as the primary somatosensory cortex. The parietal cortex is also considered important for integrating sensory information of multiple types, for example, the ability to recognize the identity of a friend and imagine his face based only on the sound of his voice. The occipital cortex is generally associated with processing light and includes what is commonly referred to as the primary visual cortex. The temporal cortex is generally associated with processing sound and includes what is commonly referred to as the primary auditory cortex. The temporal cortex is also considered important for language comprehension, translation of words into speech, sensing balance and equilibrium, and certain complex aspects of vision. The above are provided merely as examples and are not intended to represent a full listing of the many functions associated with regions of the cortex, many of which may interact and overlap in complex ways to provide these functions.

Stimulation system 10 may be used to electrically stimulate and thus provide reduced, enhanced, or otherwise modified neuroplasticity effects in the cortex, the thalamus (which among other functions provides a center for routing certain types of incoming sensory information to higher level nerve centers in the cortex), or any other suitable target nerve tissue in the brain. For example, where therapeutic electrical stimulation is directed to the somatosensory cortex for pain relief, stimulation system 10 may be used to apply additional electrical stimulation to the somatosensory cortex to reduce neuroplasticity effects associated with the therapeutic electrical stimulation. As another example, where therapeutic electrical stimulation is directed to the auditory cortex for tinnitus relief, stimulation system 10 may be used to apply additional electrical stimulation to the auditory cortex to reduce neuroplasticity effects associated with the therapeutic electrical stimulation. As another example, where a person has experienced a stroke, stimulation system 10 may provide electrical stimulation of target nerve tissue in the brain to enhance or promote neuroplasticity effects.

Other examples of altering neuroplasticity can include stimulation of a peripheral nerve as the stimulation site such that stimulation of the peripheral nerve results in alterations in a target site in the brain or spinal cord. Peripheral nerves can include, but are not limited to olfactory nerve, optic, nerve, oculomotor nerve, trochlear nerve, trigeminal nerve, abducens nerve, facial nerve, vestibulocochlear (auditory) nerve, glossopharyngeal nerve, vagal nerve, accessory nerve, hypoglossal nerve, suboccipital nerve, the greater occipital nerve, the lesser occipital nerve, the greater auricular nerve, the lesser auricular nerve, the phrenic nerve, brachial plexus, radial axillary nerves, musculocutaneous nerves, radial nerves, ulnar nerves, median nerves, intercostal nerves, lumbosacral plexus, sciatic nerves, common peroneal nerve, tibial nerves, sural nerves, femoral nerves, gluteal nerves, thoracic spinal nerves, obturator nerves, digital nerves, pudendal nerves, plantar nerves, saphenous nerves, ilioinguinal nerves, gentofemoral nerves, and iliohypogastric nerves.

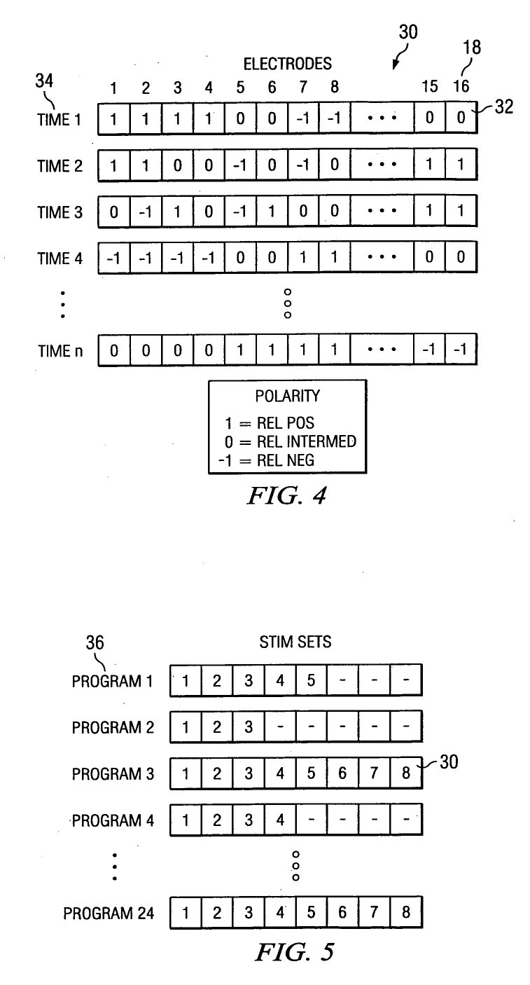

FIG. 4 illustrates an example stimulation set 30. One or more stimulation sets 30 may be provided, each stimulation set 30 specifying a number of stimulation parameters for the stimulation set 30. For example, as described more fully below with reference to FIGS. 5-6, multiple stimulation sets 30 may be executed in an appropriate sequence according to a pre-programmed or randomized stimulation program.

Stimulation parameters for a stimulation set 30 may include amplitude or intensity information, frequency information, phase information, and pulse width information for each of a series of stimulation pulses that electrodes 18 are to deliver to the target nerve tissue during a time interval during which stimulation set 30 is executed, along with a polarity 32 for each electrode 18 within each stimulation pulse. In particular embodiments in which stimulation lead 14 includes two or more electrodes 18, an electric field is generated between adjacent electrodes 18 having different polarities 32 to deliver an electrical stimulation pulse to the target nerve tissue. In particular embodiments in which stimulation lead 14 includes a single electrode 18, such as a single electrode 18 at the tip of stimulation lead 14 for example, an electric field is generated between the single electrode 18 and a terminal or other contact associated with stimulation source 12 to deliver an electrical stimulation pulse to the target nerve tissue. Stimulation parameters may also include a pulse shape, for example, biphasic cathode first, biphasic anode first, or any other suitable pulse shape.

For reducing neuroplasticity effects associated with therapeutic electrical stimulation, one or more stimulation parameters for a stimulation set 30 may be randomized or otherwise varied in any suitable manner within the time interval in which stimulation set 30 is executed, spanning one or more stimulation pulses within each stimulation pulse. For example, instead of or in addition to randomizing or otherwise varying polarities 32 for electrodes 18 as described below, the amplitude or intensity, frequency, phase information, and pulse width may be randomized or otherwise varied within predetermined ranges, singly or in any suitable combination, within each stimulation pulse. As another example, instead of or in addition to randomizing or otherwise varying polarities 32 for electrodes 18 over multiple stimulation pulses as described more fully below, the amplitude or intensity, frequency, phase information, and pulse width may be randomized or otherwise varied within predetermined ranges, singly or in any suitable combination, over multiple stimulation pulses, where the combination of stimulation parameters is substantially constant within each stimulation pulse but different for successive stimulation pulses. Such randomization or other variation of stimulation parameters for a stimulation set 30 reduces the ability of the brain to adapt to the neuroplasticity reducing electrical stimulation and dynamically reorganize itself to overcome the effects of the neuroplasticity reducing stimulation.

The polarity for an electrode 18 at a time 34 beginning a corresponding stimulation pulse or sub-interval within a stimulation pulse may be a relatively positive polarity 32, a relatively negative polarity 32, or an intermediate polarity 32 between the relatively positive polarity 32 and relatively negative polarity 32. For example, the relatively positive polarity 32 may involve a positive voltage, the relatively negative polarity 32 may involve a negative voltage, and the relatively intermediate polarity 32 may involve a zero voltage (i.e. “high impedance”). As another example, the relatively positive polarity 32 may involve a first negative voltage, the relatively negative polarity 32 may involve a second negative voltage more negative than the first negative voltage, and the relatively intermediate polarity 32 may involve a negative voltage between the first and second negative voltages. The availability of three distinct polarities 32 for an electrode 18 may be referred to as “tri-state” electrode operation. The polarity 32 for each electrode 18 may change for each of the sequence of times 34 corresponding to stimulation pulses or to sub-intervals within a stimulation pulse according to the stimulation parameters specified for the stimulation set 30. For example, as is illustrated in FIG. 6 for an example stimulation set 30 for a lead 14 with sixteen electrodes 18, the polarities 32 of the sixteen electrodes 18 may change for each of the sequence of times 34. In the example of FIG. 6, a relatively positive polarity 32 is represented using a “1,” a relatively intermediate polarity 32 is represented using a “0,” and a relatively negative polarity 32 is represented using a “−1,” although any suitable values or other representations may be used.

Where appropriate, the polarity 32 for each electrode 18 may change in a predetermined or randomized manner, randomized changes possibly being more effective with respect to any neuroplasticity reducing stimulation for reasons described above.

Where stimulation system 10 provides, in addition to therapeutic electrical stimulation, electrical stimulation to modify neuroplasticity effects associated with the therapeutic electrical stimulation, each stimulation pulse or sub-interval within a stimulation pulse may be particular to the stimulation being provided; that is, either to therapeutic electrical stimulation or to neuroplasticity reducing electrical stimulation. For example, one or more stimulation pulses or sub-intervals may be designed to provide therapeutic electrical stimulation and one or more other stimulation pulses or sub-intervals may be designed to modify neuroplasticity effects. In this case, the therapeutic stimulation pulses or sub-intervals and neuroplasticity modifying stimulation pulses or sub-intervals may be arranged temporally in any suitable manner. A therapeutic stimulation pulse or sub-interval may be separated from a successive therapeutic stimulation pulse or sub-interval by any number of neuroplasticity modifying stimulation pulses or sub-intervals and this number may be the same between each pair of therapeutic stimulation pulses or sub-intervals or may vary between each pair of therapeutic stimulation pulses or sub-intervals in a predetermined or, particularly for reducing neuroplasticity effects, a randomized manner. As another example, one or more stimulation pulses or sub-intervals may be designed to concurrently provide both therapeutic and neuroplasticity modifying electrical stimulation.

Similarly, where stimulation system 10 provides, in addition to therapeutic electrical stimulation, electrical stimulation to modify neuroplasticity effects associated with the therapeutic electrical stimulation, each stimulation set 30 may be particular to either the therapeutic electrical stimulation or the neuroplasticity modifying electrical stimulation. For example, one or more stimulation sets 30 may be designed to provide therapeutic electrical stimulation and one or more other stimulation sets 30 may be designed to modify neuroplasticity effects. In this case, the therapeutic stimulation sets 30 and neuroplasticity modifying stimulation sets 30 may be arranged temporally in any suitable manner. A therapeutic stimulation set 30 may be separated from a successive therapeutic stimulation set 30 by any number of neuroplasticity modifying stimulation sets 30 and this number may be the same between each pair of therapeutic stimulation sets 30 or may vary between each pair of therapeutic stimulation sets 30 in a predetermined or, particularly for reducing neuroplasticity effects, a randomized manner. As another example, one or more stimulation sets 30 may be designed to concurrently provide both therapeutic and neuroplasticity modifying electrical stimulation.

In addition, the amplitude or intensity, frequency, phase information, or pulse width for a stimulation set 30 may be particular to the stimulation being provided. For example, therapeutic electrical stimulation may be provided using higher amplitude electrical stimulation pulses than are used for neuroplasticity modifying electrical stimulation. In this case, the neuroplasticity modifying electrical stimulation may be below the therapeutic target threshold stimulation (i.e. below the threshold where therapeutic electrical stimulation is provided to adjust the level of activity in the target nerve tissue in the person's brain to treat the condition in the person's body). Alternatively, neuroplasticity modifying electrical stimulation may be provided using the same or a higher amplitude electrical stimulation pulses than are used for therapeutic electrical stimulation (i.e. at or above the threshold where therapeutic electrical stimulation is provided to adjust the level of activity in the target nerve tissue in the person's brain to treat the condition in the person's body). In this case, the neuroplasticity modifying electrical stimulation's primary purpose is not to produce a therapeutic effect, but rather to modify neuroplasticity. In this manner, the neuroplasticity modifying electrical stimulation could have both a therapeutic and neuroplasticity modifying effect.

FIG. 5 illustrates a number of example stimulation programs 36, each including a number of stimulation sets 30. One or more simulation programs 36 may be set up to modify neuroplasticity effects associated with therapeutic electrical stimulation of the brain. As described above, each stimulation set 30 specifies a number of stimulation parameters for the stimulation set 30. In one embodiment, within each stimulation program 36, stimulation system 10 consecutively executes the sequence of one or more stimulation sets 30 associated with stimulation program 36. The sequence may be executed only once, repeated a specified number of times, or repeated an unspecified number of times within a specified time period. For example, as illustrated in FIG. 6 for the third example stimulation program 36c including eight stimulation sets 30, each of the eight stimulation sets 30 is consecutively executed in sequence. Although the time intervals 38 (t1-t0, t2-t1, etc.) during which the stimulation sets 30 are executed are shown as being equal, a particular stimulation set 30 is preferably executed over a different time interval 38 than one or more other stimulation sets 30 according to particular needs. One or more stimulation sets 30 within at least one stimulation program 36 may be set up to provide modified neuroplasticity effects associated with therapeutic electrical stimulation of the brain.

Although stimulation system 10 is illustrated by way of example as accommodating up to twenty-four stimulation programs 36 each including up to eight stimulation sets 30, any appropriate number of stimulation programs 36 can be employed with each program including any appropriate number of stimulation sets 30. For example, in a very simple case, a single stimulation program 36 may include a single stimulation set 30, whereas in a very complex case more than twenty-four stimulation programs 36 may each include more than eight stimulation sets 30.

In one embodiment, stimulation system 10 executes only a single stimulation program 36 in response to user selection of that stimulation program for execution. In another embodiment, during a stimulation period, stimulation system 10 executes a sequence of pre-programmed stimulation programs 36 for each stimulation lead 14 until the stimulation period ends. Depending on the length of the stimulation period and the time required to execute a sequence of stimulation programs 36, the sequence may be executed one or more times. For example, the stimulation period may be defined in terms of a predetermined number of cycles each involving a single execution of the sequence of stimulation programs 36, the sequence of stimulation programs 36 being executed until the predetermined number of cycles has been completed. As another example, the stimulation period may be defined in terms of time, the sequence of stimulation programs 36 being executed until a predetermined time interval has elapsed or the patient or another user manually ends the stimulation period. Although a sequence of stimulation programs 36 is described, a single stimulation program is preferably executed one or more times during a stimulation period according to particular needs. Furthermore, each stimulation program 36 is preferably executed substantially immediately after execution of a previous stimulation program 36 or being executed after a suitable time interval has elapsed since completion of the previous stimulation program 36. Where stimulation system 10 includes multiple stimulation leads 14, stimulation programs 36 for a particular stimulation lead 14 may be executed substantially simultaneously as stimulation programs 36 for one or more other stimulation leads 14, may be alternated with stimulation programs 36 for one or more other stimulation leads 14, or may be arranged in any other suitable manner with respect to stimulation programs 36 for one or more other stimulation leads 14.

Where stimulation system 10 provides, in addition to therapeutic electrical stimulation, electrical stimulation to modify neuroplasticity effects, each stimulation program 36 may be particular to either the therapeutic electrical stimulation or the neuroplasticity modifying electrical stimulation. For example, one or more stimulation programs 36 may be designed to provide therapeutic electrical stimulation and one or more other stimulation programs 36 may be designed to modify neuroplasticity effects. In this case, the therapeutic stimulation programs 36 and the neuroplasticity modifying stimulation programs 36 may be arranged temporally in any manner. A therapeutic stimulation program 36 may be separated from a successive therapeutic stimulation program 36 by any number of neuroplasticity modifying stimulation programs 36 and this number may be the same between each pair of therapeutic stimulation programs 36 or may vary between each pair of therapeutic stimulation programs 36 in a predetermined or, particularly for reducing neuroplasticity effects, a randomized manner. As another example, one or more stimulation programs 36 may be set up to concurrently provide both therapeutic and neuroplasticity modifying electrical stimulation.

In general, each stimulation program 36 may, but need not necessarily, be set up for electrical stimulation of different target nerve tissue in a person's brain. As an example, where therapeutic electrical stimulation of target nerve tissue in a particular region 38 of the brain is desired, one or more stimulation programs 36 may be set up for therapeutic electrical stimulation of the target nerve tissue in the particular region 38 and one or more other stimulation programs 36 may be set up for electrical stimulation of the same target nerve tissue in the particular region 38 to modify neuroplasticity effects associated with the therapeutic electrical stimulation. As another example, one or more stimulation programs 36 may be set up for therapeutic electrical stimulation of target nerve tissue in a particular region 38 of the brain and one or more other stimulation programs 36 may be set up for electrical stimulation of different nerve tissue in either the same region 38 or in a different region 38 of the brain to modify neuroplasticity effects associated with the therapeutic electrical stimulation.

As described above, in one embodiment, the nature of any neuroplasticity reducing electrical stimulation may be varied more or less continually, whether in a predetermined or randomized manner, to reduce, prevent, delay, enhance, promote, or otherwise control the ability of the brain to adapt to the neuroplasticity reducing electrical stimulation and dynamically reorganize itself accordingly. In a more particular embodiment, where the neuroplasticity reducing electrical stimulation is provided concurrently with therapeutic electrical stimulation, the neuroplasticity reducing electrical stimulation may be randomized or otherwise varied about the therapeutic electrical stimulation to achieve this result. In essence, the randomized or otherwise varied neuroplasticity reducing electrical stimulation makes it more difficult for the brain to dynamically reorganize itself to overcome the effects of the therapeutic electrical stimulation.

Any suitable circuitry within stimulation source 12 can be employed for generating and transmitting electrical stimulation pulses for electrically stimulating target nerve tissue in a person's brain, spinal cord, or peripheral or other nerve to reduce, enhance, or otherwise modify neuroplasticity effects, whether separate from or concurrently with the therapeutic electrical stimulation. Example circuitry that may be used is illustrated and described in U.S. Pat. No. 6,609,031 B1, which is hereby incorporated by reference herein as if fully illustrated and described herein.

Although embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture,-compositions of matter, means, methods, or steps.

What is claimed is:

1. A method for effecting neuroplasticity in brain tissue of a patient using neurostimulation, comprising:
receiving a selection of a first electrode, by a programming device, for stimulation of a first location associated with brain tissue of the patient, wherein a neuronal function previously occurred at the first location before impairment of the neuronal function;
receiving a selection of a second electrode, by the programming device, for stimulation of a second location associated with brain tissue of the patient, wherein the second location is a location that is capable of at least partially assuming performance of the neuronal function;
programming, by the programming device, an implantable pulse generator (IPG) to operate according to programmable parameters, the programmable parameters including parameters defining stimulation pulses to be applied via the second electrode that encourage subsequent performance of the neuronal function at the second location by promoting neuroplasticity; and
activating the IPG to stimulate the first and second locations according to the programmable parameters.

2. The method of claim 1 wherein the first and second electrodes are electrodes of a percutaneous lead.

3. The method of claim 1 wherein the first and second electrodes are electrodes of a paddle lead.

4. The method of claim 1 wherein the first location is a location of the brain damaged by a stroke.

5. The method of claim 1 wherein the first location is a location of the brain damaged by disease.

6. The method of claim 1 wherein the first location is a location of the brain damaged by physical injury.

7. The method of claim 1 wherein the first and second locations are within the same region of the patient's brain selected from the list consisting of: motor cortex, somatosensory cortex, visual cortex, and auditory cortex.

8. The method of claim 1 wherein the generated parameters include a first stimulation set defining stimulation for the first location and a second stimulation set defining stimulation for the second location.

9. The method of claim 1 wherein the programming device includes at least one stimulation set stored in memory for selection by a user that includes parameters adapted to promote neuroplasiticity at a given location without otherwise impairing neuronal functions at the given location.

10. The method of claim 1 wherein stimulation pulses applied to the second location are below a therapeutic threshold.

11. A system for effecting neuroplasticity in brain tissue of a patient using neurostimulation, comprising:
an implantable pulse generator (IPG) for generating electrical stimulation;
a first electrode electrically coupled to the IPG for delivering electrical pulses to a first location associated with brain tissue of the patient;
a second electrode electrically coupled to the IPG for delivering electrical pulses to a second location associated with brain tissue of the patient;
the IPG storing a first set of parameters defining pulses above a therapeutic threshold for application to the first location; and
the IPG storing a second set of parameters defining pulses below a therapeutic threshold for application to the second location.

12. The system of claim 11 wherein the first location is a location where a neuronal function previously occurred before impairment of the neuronal function in the patient.

13. The system of claim 12 wherein the second location is a location that is capable of assuming performance of the neuronal function and the second set of parameters are selected to encourage performance of the neuronal function at a second location by promoting neuroplasticity.

14. The system of claim 11 further comprising:
a programming device for controlling parameters of the IPG, wherein the programming device stores parameters, below the therapeutic threshold and suitable for promoting neuroplasticity in the patient, for selection by a user for downloading to the IPG.

15. The system of claim 11 wherein the first and second electrodes are electrodes of a percutaneous lead.

16. The system of claim 11 wherein the first and second electrodes are electrodes of a paddle lead.

17. The system of claim 11 wherein the first location is a location of the brain damaged by a stroke.

18. The system of claim 11 wherein the first location is a location of the brain damaged by disease.

19. The system of claim 11 wherein the first location is a location of the brain damaged by physical injury.

20. The system of claim 11 wherein the first and second locations are within the same region of the patient's brain selected from the list consisting of: motor cortex, somatosensory cortex, visual cortex, and auditory cortex.
